(19) United States
(12) Patent Application Publication
(10) Pub. No.: US 2017/0157005 A1
(43) Pub. Date: Jun. 8, 2017

(54) Novel Anti-Wrinkle and Anti-Aging Nano Formulations and Method of Preparation using Novel Nano Co-Delivery System

(71) Applicant: Anamaria Ioana Orza, Atlanta, GA (US)

(72) Inventor: Anamaria Ioana Orza, Atlanta, GA (US)

(73) Assignee: IndagoMed. LLC, Atlanta, GA (US)

(21) Appl. No.: 15/334,310

(22) Filed: Oct. 26, 2016

(51) Int. Cl. A61K 8/25; A61K 8/11; A61K 8/92; A61K 8/73; A61K 8/44; A61K 8/97; A61K 8/67; A61Q 19/00; A61Q 19/08; A61K 8/64

(52) U.S. Cl. CPC A61K 8/25; A61Q 19/08; A61K 8/11; A61K 8/922; A61K 8/64; A61K 8/44; A61K 8/97; A61K 8/678; A61K 8/676; A61Q 19/007; A61K 8/735

(57) ABSTRACT

The invention relates to new anti-wrinkle and anti-aging nanoformulations, composed of non-toxic mesoporous silica nanoparticles, natural plant extracts (as the case may be: pomegranate oil, fennel oil, rosemary oil, chamomile oil, jojoba oil, rosehip oil), biological active agents (acetyl hexapeptide-8, aspartic acid), vitamins and others. Additionally, the present invention relates a new method for preparing the nanoformulations by co-encapsulating its active agents within a multilayer nanocarrier, thereby, enhancing their delivery through the skin barrier and their controlled accumulation at the desired site.

In certain embodiments, the active molecules are co-incorporated into an inner polymeric shell (silica derivatives) and onto its surface, followed by, the protection of an outer layer of chitosan-TAT (GRKKRRQRRRPQ). The active ingredients are crossed through the skin barrier using the disclosed delivery system to promote cell growth, skin rejuvenation and wrinkle removal.

### BACKGROUND OF THE INVENTION

To date, anti-wrinkle and anti-aging creams are fabricated by thousands of companies and gave birth to a multi-billion industry. Unfortunately, a high majority of cosmetics industry players use a variety of toxic synthetic chemicals into their personal care products. Toxic chemicals that act as: (1) active substances or (2) chemical enhancers, stabilizers, etc., are found in everything from anti-aging moisturizers, body lotions to shampoo and shaving cream. The risk of using products containing such chemicals is devastating, since they can lead to diseases such as: cancer, infertility, birth defects, allergies, etc. Even the well-known and some luxurious brands such as: Chanel, Sisley, Dior, Estee Lauder, Aveeno, Chanel, L'oreal are using life-threatening chemical in their products composition. For instance, products that contain:
(1) Placental extract and Polycyclic Aromatic Hydrocarbons (PAHs) are highly carcinogen. Examples of Brands using placental extract: JYP New Zealand, HornePeel, Rebirth, etc. Moreover, Polycyclic Aromatic Hydrocarbons (PAHs) have shown increase risk for breast cancer. Examples of Brands using PAHs: L'Oreal, Maybelline NY, etc¹
(2) Retinyl palmitate, retinyl propionate, retinyl linoleate and other retinyl complexes develop reproductive toxicity, biochemical or cellular level changes, cancer, organ system toxicity (non-reproductive). Example of brands: Olay Age Defying Anti-Wrinkle Eye Cream², L'Oreal Revitalift³, Gamier Nutritioniste Ultra-Lift Anti-Wrinkle Firming Night Cream.
(3) Parabenes and its complexes cause high endocrin disruptions and are possible carcinogenic.⁴ Example of brands using parabenes in their composition: AmLactin,⁵ Olay, Sisley, L'oreal, Neutrogena, Avene, RoC, La Roche, Gamier⁶.
(4) Fragrance causes allergies/immunotoxicity, irritation (skin, eyes, or lungs), organ system toxicity (non-reproductive), and ecotoxicology. Used by: Chanel (Chanel No 5⁷, Chanel Les Beiges All In One Healthy Glow Fluid⁸), Dior (Christian Dior Hydra Life BB Creme Enhancing Moisturizer)⁹, Este Lauder (Estee Lauder Daywear Anti-oxidant Beauty Benefit BB Creme).¹⁰
(5) Oxybenzone is a sunscreen ingredient associated with photoallergic reactions and it's possible carcinogen^{11,12}. It has been noted to induce biochemical or cellular level changes, allergies/immunotoxicity, endocrine disruption, developmental/reproductive toxicity, organ system toxicity (non-reproductive). Brands using this chemical are: Neutrogena, Physicians Formula, RoC, Pond's,¹³
(6) Other chemicals such as: Sodium Lauryl Sulfate₁₄, propenyl glycol¹⁵, Diethanolamine¹⁶, Hexachlorophene, Mercury compounds, Chlorofluorocarbon propellants¹⁷, Hydroquinone etc., are all of a high health concern.

Apart from the toxic chemicals, another major concern is over the incorporation of nanotechnology in skin care. Using nanotechnology in various skin care compositions, novel enhanced properties such as: better skin penetration, transparency, unique texture, color, solubility, and enhanced UV protection are gained. However, high toxicity concerns result when nanoparticles pass into the blood stream. It has been reported that the subcutaneously exposure of TiO₂ nanoparticles produce brain damage and reduces the sperm production in male. ¹⁸ Other researchers reported the toxicity of TiO₂ and ZnO NPs.¹⁹⁻²³ Moreover, silver nanoparticles that are used for their antimicrobial activity show both keratinocytes and fibroblasts apoptosis.²⁴ Other nanoparticles that were applied in skin care were: gold nanoparticles, metal oxides, liposomes, nanocapsule, solid lipid nanoparticles, nanocrystals, dendrimers, cubosomes, niosomes, and fullerene. From the large number of reports on the toxicity of nanomaterials, it can be concluded that the toxicity is greater influenced by the: (a) physicochemical properties of the nanomaterial; (b) morphology; (c) chemical composition; (d) size; (e) shape and (f) surface chemistry. ²⁵⁻²⁹

Therefore, it is necessary to avoid the use of both toxic synthetic chemicals and metal/metal oxide nanoparticles in the formulations of skin care products. Moreover, the aforementioned nanoparticles have no specific regulations regarding their safety assessment. Thus, there is a rising demand driven by natural skin care products with no unnecessary toxic additives. However, It's not enough to offer beauty made with sustainably and natural sourced ingredients; the ingredients have to proven efficiency.

Nevertheless, natural skin care products become very attractive and important in cosmetic industry and are expected to offer better consumer compliance. However, no suitable and efficient marketed anti-wrinkle and anti-aging cream has meet this ever increase need, most likely because: 1^{st}, the active molecules are very sensitive to the environment, they are unstable to temperature, pH, light and oxidation. Moreover, other additives can denature and decrease their activity; and 2^{nd}, specific active molecules from plant extracts or oils have poor penetration of the skin barrier; therefore it's hard to get across the skin. Moreover, these substances may undergo undesired reactions that lead to the reduction or loss of their effectiveness or even lead to the degradation of the cosmetic product.

The anti-wrinkle and anti-aging nanoformulations disclosed in the present invention, overcome the obstacles presented above by offering non-toxic ingredients with novel, enhanced properties and new superior skin penetration mechanism that prompts cell growth, skin rejuvenation and wrinkle removal. It has showed an efficiency of 60% wrinkle removal when using it twice a day for a period of 30 days.

The non-toxic ingredients are comprised of: natural plant (tragacanth) natural oils extracts (as the case may be: pomegranate oil, fennel oil, citrus oil, rosemary oil, chamomile oil, jojoba oil, rosehip oil), biological active ingredients (acetyl hexapeptide-8, aspartic acid), minerals (Zn Mg Silicates), Vitamins (as the case may be—Vitamin C, Vitamin E, Vitamin K, Omega 3) and other natural additives (glutanthione, hyaluronic acid). Moreover, the new superior delivery mechanism results from using the nanotechnology benefits, such as: the active ingredients are co-encapsule in a layer-by-layer delivery system offering enhanced penetration, lower active molecules applied to the skin but more efficient results, and new visual tactical properties. The co-delivery system can contain two or more agents, such as: (1) Silica shells that have been encapsulated with targeted oils, mineral, vitamins and other active ingredients; (2) anti-wrinkle and anti-aging peptide Argieline-8 that is functionalized on the surface of the shell; (3) a shell of chitosan-TAT. Wherein, Argireline-8 acts in similar way as botulinum neurotoxins (Botox®, Allergan, Irvine, Calif.) causing muscle paralysis by inhibiting activity in the presynaptic neuronal exocytosis machinery.³⁰ The oil extracts, minerals and vitamins have a synergetic effect and are used for hydration, wrinkle activity, anti-oxidant properties, etc. Moreover, the presence of chitosan has a dual activity, both as an antiseptic and for co-encapsulation of active compounds. Furthermore, the TAT peptide is conjugated on the surface of the nanoparticles to ensure a better internalization of the nanoparticles through the cell membrane into the cytosol.

Generally, the nano-formulations will be treated in such a way to prevent contamination with various viruses and bacteria and also may be stabilized by modifying the pH, ionic strength. In a typical formulation, the present invention will utilize:
Nano encapsulated active compounds: Specific oils (as the case may be), the peptide acetyl hexapeptide-8, aspartic acid are used herein to improve the appearance of the skin by decreasing the wrinkles and conferring the skin rejuvenation. The amount of active molecules and oils in the final composition vary from about 0.1% (v/w) to about 30% (v/w) and most preferable from about 5% (v/w) to about 15% (v/w).
Antimicrobial agents: green tea extracts, they are added in a concentration between 0.002% to about 0.06% but most preferable 0.0125% to about 0.025%
Vitamins: Vitamin C (ascorbic acid), Vitamin D, Vitamine K, Vitamine E, Vitamin Q and a mixture of those are used in a concentration range between 0.0005% (v/w) to about 0.01% (v/w) and most preferable from about 0.001% (v/w) to about 0.005% (v/w).
Minerals: In accordance with the present invention, Mg silicate and Zn are used in a concentration rage between 0.0005% (v/w) to about 0.01% (v/w) and most preferable from about 0.001% (v/w) to about 0.005% (v/w).
Anionic Surfactants in a concentration range varying from 0.005% (v/w) to about 30% (v/w) and more preferably from about 0.1% (v/w) to about 15% (v/w).
Moisturizer oils: oil or an oil mixture (presented above as the case may be) that will deliver and also promote the retention of water of the surface of the skin. The oils that are defined, as moisturizer will comprise other properties such as: emollients. The moisturizer will comprise from about 1% (v/w) to about 60% (v/w), and most preferable from about 5% (v/w) to about 40% (v/w).
Fragrances: In the examples section, Example 1-5, citric oil is used as a fragrance. Other natural oils which may be formulated in accordance with the present invention such as: coconut, lavender, jasmine, floral, rose, herbal, lily, orange, etc Typically, the fragrances comprises between 0.005%-10% (New), more preferable between 0.5% (v/w) to about 5% (v/w). Note that the disclosed oils properties might act in a synergetic effect such as: fragrance, moisture, anti-oxidant properties, etc
Preservatives: molecules used to prevent oxidative reaction here in we used Vitamin C, Optiphen Preservativ etc., in a concentration of 0.01% (v/w) to 2% (v/w) most preferable 0.05% (v/w) to about 1% (v/w). Additionally, molecules that prevent physically stability, herein glycerol, that is used in a concentration of 0.01% (v/w) to 2% (v/w) and most preferable 0.05% (v/w) to about 1% (v/w).

### DETAILED DISCLOSURE OF THE INVENTION

The invention describes both new highly efficient anti-wrinkle nanoformulations and also new method to deliver active components across the skin barrier by using a layer-by-layer co-delivery nano-system. The active ingredients disclosed herein, are both natural oils (as the case may be: pomegranate oil, fennel oil, citrus oil, rosemary oil, chamomile oil, jojoba oil, rosehip oil), Vitamins (as the case may be—Vitamin C, Vitamin E, Vitamin K, Omega 3), Minerals (Zn, Mg Silicates) and Argireline (Ac-Glu-Glu-Met-Gln-Arg-Arg-NH2), Aspartic acid.

The high anti-wrinkle efficiency is a result of a synergetic effect of the active components, concentrations and co-delivery system. The multi shell co-delivery system is such designed such as to have enough solubility in the lipid domain of the stratum corneum, while still having sufficient hydrophilic nature to allow partitioning into the skin inner layers for an optimum delivery. Furthermore, the nanoparticles penetration is enhanced by the presence of TAT peptide (Human Immunodeficiency Virus (HIV) Trans-activator of transcription) on the nanoparticles surface, that is increasing the zeta potential of the nanoparticles (from without TAT 5.36±3.72 to 11.3±2.1 with TAT) and therefore, increasing both the adhesion and lysosome escape of the nanoparticles into cytosol.^{31,32} The presence of cationic charge on the surface of the nanoparticles has been reported to interact with the negative charge of the endosomal membrane resulting in destabilization of the membrane and the nanoparticles escape into the cytosol.^{33,34}

The multi layer nanoparticles-core-shell co-system can consist (but not limited) of biodegradable and biocompatible polymers and sugars (casein, polysaccharides, chitosan, etc), consisting of smaller shells nanoparticles (silica, gold shell, etc) and co-encapsulated active ingredients and CPP (cell penetrating peptides). The co-system nanoparticles have a good stability, even when very mild conditions are used to encapsulate the desired substances. In accordance with the present invention, the nanoparticles penetration and accumulation at the desired site is more superior and follows the mechanism: (a) hair follicular channels, intracellular space and intercellular space or membranes. The encapsulated compounds are released gradually in various layers of the penetration pathways.

The co-system nanoparticles size can be controlled at: 30 nm, 40 nm, 50 nm, 60 nm or so. The encapsulated nanoparticles co-system is used in a nanoemulsion as a cream in order to serve the invention purpose, such as: to promote cell growth, skin rejuvenation and wrinkle removal. Note that, the delivery system can be used to serve various other purpose such as: delivery of other active compounds to fight: eczemas, psoriasis, wound healing, burn skin healing, skin whitening, firming-up.

The present invention gains superior results based on both its composition and delivery method. The disclosed delivery system and method has the ability to incorporate many active and sensitive ingredients using a new co-encapsulation method. Using this multi layer nano-delivery, lower quantities of active compounds are transported through the skin, at the targeted site with a controlled release thus, offering a prolong effect and lower toxicity.^{35,36} Additionally, it offers isolation and protection from the environment, therefore, improvements in stability, efficiency but also improvements in tactile and the visual appearance of the products. The method it's offering new products, in response to human needs and desires.

To date, liposomes technology have been wildly reported and use in cosmetics and medicine for incorporation of various compounds, however, to the manufacture process that requires temperatures higher than 50° C., some active components and other biological active molecules are denaturized. Other common nanoparticles used in cosmetic are polysaccharides (gums, starch, cellulose, cyclodextrines and chitosan)^{37,38}, proteins (gelatin, casein and soy proteins), lipids³⁹ (waxes, paraffin and oils)⁴⁰ and synthetic polymers [acrylic polymers, polyvinyl alcohol and poly(vinylpyrrolidone)] and biodegradable polymers.^{41,42} Additionally, polymers such as: silicates,⁴³ clays and polyphosphates were reported. Moreover, other simple and single layer nanoparticles (ex: synthetic polymers) of a certain size (100-200 nm) have difficulties to cross the multiple layers of skin and cells through lipophilic and hydrophilic membrane. Our co-system gain superiority compared with the reported once based on its small sizes (between 20 nm-70 nm) and its capacity to co-deliver in an efficient way two or more sensitive molecule of high molecular weight.

### BEST MODE FOR CARRYING OUT THE INVENTION

### EXAMPLES

### Example 1

### Preparation of a Cosmetic Anti-Wrinkle and Anti-Aging Cream A. Using a Nano Layer-By-Layer Delivery System

Step 1. Design and Synthesis of a Layer-By-Layer Co-Delivery System (chi-MSNs)

Encapsulation of the active ingredients into the Silica Shells (MSNs)

Jojoba oil (60%), Rosehip oil (39.9%), vitamin E (0.05%) and Omega 3 (0.05%) were mixed at room temperature for 30 min to form an emulsion. After, we added dropwise under magnetic stirring both the DEODMS (final concentrations ranging from 0.005 to 0.035 mol dm⁻³) and TEOS (0.018 mol dm⁻³). The formed dispersion was stirred for 1 h and then left to stand for 48 h so the reaction is completed. The dispersion was then purified using centrifugation.

Co-Encapsulation of MSNs with other active ingredients and its TAT-chitosan surface modification (chi-MSNs)

The as prepared MSNs were then functionalized with argieline-8 peptide and vitamin C followed by the addition of the TAT-chitosan polymer such as:

Preparation of TAT-Chitosan

First, TAT-chitosan conjugation was performed as follows: 1 ml of 1% chitosan in 1% HCl solution containing by adjusting the pH=6 were mixed with 200 mM EDAC and 200 mM NHSS at room temperature for 5 min. After, 5 mg of HIV-1 Tat peptide was added to the chitosan solution and stirred for 4 h at room temperature. Next, 1 mg of tris(2-carboxy-ethyl)phosphine hydrochloride was added and the solution was then left undisturbed for 30 min. The sample was then purified using centrifugation.

Co-Encapsulated Process

In a typical reaction, 0.25 g of previously prepared MSNs was dispersed in 5 mL anhydrous ethanol and 0.1 mg argieline-8 peptide and 0.06 mg Vitamin C was added and the suspension was then shacked overnight at room temperature. Afterwards, 10 ml TAT-chitosan was added into the solution and the reaction was continued for another 8 h. The layer-by-layer system was then purified through centrifugation and washing with DI water. The nanoparticles can be lyophilized and used when needed.

Step. 2. The Preparation of a Cosmetic Anti-Wrinkle and Anti-Aging Cream A. Using the Layer-By-Layer Delivery System

The chi-MSNs phase was used to formulate a skin care elegant product as follows: The water-soluble ingredient: tragacanth plant extract (0.65%) was dissolved under magnetic stirring of 150 RPM at room temperature in 300 ml H₂O. The emulsion was then stored for 10 days at 5° C. in CO₂. The emulsion formed a homogeneous east cream (28.8%), in which, glycerin (2%) and Argieline-8 (10%), hyaluronic acid (0.2%), MG, Al Silicate (4%), was added while mixing and then the suspension was heated at moderate agitation at 75° C. Subsequently, an oil phase containing the Chi-MSNs co-system nanoparticles (5%) along with Castrol oil (20%), Grape Seed Oil (5%), Jojoba oil (2%), Citrus oil (5%), were also heated at 75° C. then added on the water phase at vigorous agitation for 30 min. The resulting emulsion was then gradually cooled down. Other Oil formulations and minerals can be added into the emulsion. The mixture is then allowed to cool at room temperature

### Example 2

### Preparation of a Cosmetic Anti-Wrinkle and Anti-Aging Serum A. Using a Nano Layer-By-Layer Delivery System

Step 1. Design and Synthesis of a Layer-By-Layer Co-Delivery System (chi-MSNs)

Encapsulation of the active ingredients into the Silica Shells (MSNs)

The active ingredients such as: (1) Rosehip oil (40%) and (2) Argieline (60%), were mixed at room temperature for 30 min to form an emulsion. After, we added dropwise under magnetic stirring both the DEODMS (final concentrations ranging from 0.005 to 0.035 mol dm⁻³) and TEOS (0.018 mol dm⁻³). The formed dispersion was stirred for 1 h and then left to stand for 48 h so the reaction is completed. The dispersion was then purified using centrifugation

Co-Encapsulation of MSNs with other active ingredients and its TAT-chitosan surface modification (chi-MSNs)

The as prepared MSNs were then functionalized with argieline-8 peptide and vitamin C followed by the addition of the TAT-chitosan polymer.

Preparation of TAT-Chitosan

First, TAT-chitosan conjugation was performed as follows: 1 ml of 1% chitosan in 1% HCl solution containing by adjusting the pH=6 were mixed with 200 mM EDAC and 200 mM NHSS at room temperature for 5 min. After, 5 mg of HIV-1 Tat peptide was added to the chitosan solution and stirred for 4 h at room temperature. Next, 1 mg of tris(2-carboxy-ethyl)phosphine hydrochloride was added and the solution was then left undisturbed for 30 min. The sample was then purified using centrifugation.

Co-Encapsulated Process

In a typical reaction, 0.25 g of previously prepared MSNs was dispersed in 5 mL anhydrous ethanol and 0.3 mg argieline-8 peptide and 0.06 mg Vitamin C was added and the suspension was then shacked overnight at room temperature. Afterwards, 10 ml TAT-chitosan was added into the solution and the reaction was continued for another 8 h. The layer-by-layer system was then purified through centrifugation and washing with DI water. The nanoparticles can be lyophilized and used when needed.

Step. 2. The Preparation of a Cosmetic Anti-Wrinkle and Anti-Aging Cream Serum Using the Layer-By-Layer Delivery System

The chi-MSNs phase was used to formulate a skin care elegant product as follows: The water-soluble ingredient: tragacanth plant extract (0.65%) was dissolved under magnetic stirring of 150 RPM at room temperature in 300 ml H₂O. The emulsion was then stored for 10 days at 5° C. in CO₂. The emulsion formed a homogeneous east cream (33.75%), in which, glycerin (2%) and Argieline-8 (25%), hyaluronic acid (0.2%), Mg Silicate (4%), Vitamin C (0.05%) was added while mixing and then the suspension was heated at moderate agitation at 75° C. Subsequently, an oil phase containing the Chi-MSNs co-system nanoparticles (15%) along with Rosehip oil (20%) were also heated at 75° C. then added onto the water phase at vigorous agitation for 30 min. The resulting emulsion was then gradually cooled down. The mixture is then allowed to cool at room temperature.

### Example 3

### Preparation of a Skin Care Anti-Wrinkle and Anti-Aging Eye Cream Using a Nano Layer-By-Layer Delivery System

Step 1. Design and Synthesis of a Layer-By-Layer Co-Delivery System (chi-MSNs)

Encapsulation of the active ingredients into the Silica Shells (MSNs)

Fennel Essential oil (33.3%), Rosemary oil (33.3%), Chamomile oil (33.3%) were mixed at room temperature for 30 min to form an emulsion. After, we added dropwise under magnetic stirring both the DEODMS (final concentrations ranging from 0.005 to 0.035 mol dm⁻³) and TEOS (0.018 mol dm⁻³). The formed dispersion was stirred for 1 h and then left to stand for 48 h so the reaction is completed. The dispersion was then purified using centrifugation.

Co-Encapsulation of MSNs with other active ingredients and its TAT-chitosan surface modification (chi-MSNs)

The as prepared MSNs were then functionalized with argieline-8 peptide and vitamin C followed by the addition of the TAT-chitosan polymer.

Preparation of TAT-Chitosan

First, TAT-chitosan conjugation was performed as follows: 1 ml of 1% chitosan in 1% HCl solution containing by adjusting the were mixed with 200 mM EDAC and 200 mM NHSS at room temperature for 5 min. After, 5 mg of HIV-1 Tat peptide was added to the chitosan solution and stirred for 4 h at room temperature. Next, 1 mg of tris(2-carboxy-ethyl)phosphine hydrochloride was added and the solution was then left undisturbed for 30 min. The sample was then purified using centrifugation.

Co-encapsulated Process

In a typical reaction, 0.25 g of previously prepared MSNs was dispersed in 5 mL anhydrous ethanol and 0.3 mg argieline-8 peptide was added and the suspension was then shacked overnight at room temperature. Afterwards, 10 ml TAT-chitosan was added into the solution and the reaction was continued for another 8 h. Further, 5 mg of Argieline was added onto the nanocapsule. The layer-by-layer system was then purified through centrifugation and washing with DI water. The nanoparticles can be lyophilized and used when needed.

Step. 2. The Preparation of a Cosmetic Anti-Wrinkle and Anti-Aging Eye Cream A. Using the Layer-By-Layer Delivery System

The chi-MSNs phase was used to formulate a skin care elegant product as follows: The water-soluble ingredient: tragacanth plant extract (0.65%) was dissolved under magnetic stirring of 150 RPM at room temperature in 300 ml H₂O. The emulsion was then stored for 10 days at 5° C. in CO₂. The emulsion formed a homogeneous east cream (40%), in which, glycerin (2%) and Argieline-8 (30%) were added while mixing and then the suspension was hated at moderate agitation at 75° C. Subsequently, an oil phase containing the Chi-MSNs co-system nanoparticles (5%) along with Rosemary oil (6%), Chamomile Oil (6%), Fennel oil (6%), Citrus oil (5%), were also heated at 75° C. then added on the water phase at vigorous agitation for 30 min. The resulting emulsion was then gradually cooled down. Other Oil formulations and minerals can be added into the emulsion. The mixture is then allowed to cool at room temperature

### Example 4

### Preparation of a Cosmetic Anti-Wrinkle and Anti-Aging Mask Using a Nano Layer-By-Layer Delivery System

Step 1. Design and Synthesis of a Layer-By-Layer Co-Delivery System (chi-MSNs)

Encapsulation of the active ingredients into the Silica Shells (MSNs)

Pomegranate oil (60%), vitamin A (0.05%), Vitamin E (0.05%), Vitamin K (0.05%), and Vitamin D (0.05%) and Omega 3 (0.05%) were mixed at room temperature for 30 min to form an emulsions. After, we added dropwise under magnetic stirring both the DEODMS (final concentrations ranging from 0.005 to 0.035 mol dm⁻³) and TEOS (0.018 mol dm⁻³). The formed dispersion was stirred for 1 h and then left to stand for 48 h so the reaction is completed. The dispersion was then purified using centrifugation.

Co-Encapsulation of MSNs with other active ingredients and its TAT-chitosan surface modification (chi-MSNs)

The as prepared MSNs were then functionalized with argieline-8 peptide and vitamin C followed by the addition of the TAT-chitosan polymer.

Preparation of TAT-Chitosan

First, TAT-chitosan conjugation was performed as follows: 1 ml of 1% chitosan in 1% HCl solution containing by adjusting the pH=6 were mixed with 200 mM EDAC and 200 mM NHSS at room temperature for 5 min. After, 5 mg of HIV-1 Tat peptide was added to the chitosan solution and stirred for 4 h at room temperature. Next, 1 mg of tris(2-carboxy-ethyl)phosphine hydrochloride was added and the solution was then left undisturbed for 30 min. The sample was then purified using centrifugation.

Co-Encapsulated Process

In a typical reaction, 0.25 g of previously prepared MSNs was dispersed in 5 mL anhydrous ethanol and 0.1 mg argieline-8 peptide, 0.06 mg Vitamin C and 0.0065 of vitamin B complex was added and the suspension was then shacked overnight at room temperature. Afterwards, 10 ml TAT-chitosan was added into the solution and the reaction was continued for another 8 h. The layer-by-layer system was then purified through centrifugation and washing with DI water. The nanoparticles can be lyophilized and used when needed.

Step. 2. The Preparation of a Cosmetic Anti-Wrinkle and Anti-Aging Cream A. Using the Layer-By-Layer Delivery System

The chi-MSNs phase was used to formulate a skin care elegant product as follows: The water-soluble ingredient: tragacanth plant extract (0.65%) was dissolved under magnetic stirring of 150 RPM at room temperature in 300 ml H₂O. The emulsion was then stored for 10 days at 5° C. in CO₂. The emulsion formed a homogeneous east cream (40%), in which, glycerin (2%) and Argieline-8 (10%), hyaluronic acid (0.2%), Zn, Mg Silicate (4%), was added while mixing and then the suspension was heated at moderate agitation at 75° C. Subsequently, an oil phase containing the Chi-MSNs co-system nanoparticles (5%) along with Pomegranate oil (25%), Jojoba oil (2%), Citrus oil (5%), were also heated at 75° C. then added on the water phase at vigorous agitation for 30 min. The resulting emulsion was then gradually cooled down. Other Oil formulations and minerals can be added into the emulsion. The mixture is then allowed to cool at room temperature

### BIBLIOGRAPHY

1. https://www.ewg.org/skindeep/browse.php?notold=1&containing=704787&&showmore=products&start=0]
2. https://www.ewg.org/skindeep/product/465864/Olay_Age_Defying_Anti-Wrinkle_Eye_Cream/
3. ¹https://www.ewg.org/skindeep/product/596247/L%27Oreal_Paris_Revitalift_AntiWrinkle_%2B_Firming%2C_Face%3B%3BNeck_Contour_Cream/
4. Boberg J, Taxvig C, Christiansen S, Hass U. Possible endocrine disrupting effects of parabens and their metabolites. Reprod Toxicol. 2010 Sep; 30(2):301-12. Epub 2010 Apr 8.; Byford J R., Shaw L E, Drew M G, Pope G S, Sauer M J, Darbre P D. Oestrogenic activity of parabens in MCF7 human breast cancer cells. J Steroid Biochem Mol Biol. 2002 Jan; 80(1):49-60.
5. https://www.ewg.org/skindeep/product/531988/AmLactin_Ultra_Hydrating_Body_Cream%2C_Fragrance_Free/
6. https://www.ewg.org/skindeep/browse.php?ingred06=705335&category=anti-aging&containing=705335&ingred06=705335&&showmore=products&start=40
7. ¹https://www.ewg.org/skindeep/ingredient/702512/FRAGRANCE/
8. https://www.ewg.org/skindeep/product/620292/Chanel_Les_Beiges_All-inOne_Healthy_Glow_Fluid%2C_SPF_15_%282015_formulation%29/
9. https://www.ewg.org/skindeep/product/669267/Dior_Hydra_Life_Water_BB_Creme%2C_Golden_Peach%2C_SPF_30/
10. https://www.ewg.org/skindeep/product/514340/Estee_Lauder_Daywear_Anti-oxidant_Beauty_Benefit_BB_Creme%2C_SPF_35_%282013_formulation%29/
11. Benson H A. 2000. Assessment and clinical implications of absorption of sunscreens across skin. Am J Clin Dermatol 1(4): 217-224.
12. European Commission. 2013. Cosing, the European Commission database with information on cosmetic substances and ingredients. Accessed on Mar. 1, 2016 at http://ec.europa.eu/consumers/cosmetics/cosing/
13. https://www.ewg.org/skindeep/browse.php?category=antiaging&ingred06=704372
14. http://www.healthy-communications.com/journal_of_the_american_college_.html
15. http://www.healthy-communications.com/msdspropyleneglycol.html
16. http://ntp.niehs.nih.gov/results/pubs/longterm/reports/longterm/tr400499/abstracts/tr478/index.html
17. http://www.healthy-communications.com/listofcarcinogens_from_the_usgov.htm
18. K. Takeda, K.-I. Suzuki, A. Ishihara et al., “Nanoparticles transferred from pregnant mice to their offspring can damage the genital and cranial nerve systems,” Journal of Health Science, vol. 55, no. 1, pp. 95-102, 2009.
19. C. Contado, A. Pagneni, Anal. Chem. 2008, 80, 7594. A.-S. Foliate , J.-F. Masfaraud, E. Bigorgne, J. Nahmani, P. Chaurand.
20. C. Lorenz, K. Tiede, S. Tear, A. Boxall, N. von Goetz, K. Hungerbuehler, Int. J. Occup. Environ. Health 2010, 16, 406.
21. R. Landsiedel, L. Ma-Hock, B. Van Ravenzwaay, M. Schulz, K. Wiench, S. Champ, S. Schulte, W. Wohlleben, F. Oesch, Nanotoxicology, 2010, 4, 364.
22. L. Zhang, R. Bai, B. Li, C. Ge, J. Du, Y. Liu, L. Le Guyader, Y. Zhao, Y. Wu, S. He, Y. Ma, C. Chen, Toxicol. Lett. 2011, 207, 73.
23. V. Sharma, S. K. Singh, D. Anderson, D. J. Tobin, A. Dhawan, J. Nanosci. Nanotechnol. 2011, 11, 3782.
24. C. Botta, J. Labile, J. Rose, J.-F. Ferard, S. Cotelle, Environ. Pollut. 2011, 159, 2515
25. V. K. M. Poon and A. Burd, “In vitro cytotoxity of silver: implication for clinical wound care,” Burns, vol. 30, no. 2, pp. 140-147, 2004
26. I. L. Hsiao, Y.-J. Huang, Total Environ. 2011, 409, 1219.
27. X Huang, L. Li, T. Liu., N. Hao, H. Liu, D. Chen, F. Tang, ACS Nano 2011, 5, 5390
28. Y. Qiu, Y. Liu, L. Wag, L. Xu, R. Bai, Y. Ji, X. Wu, Y. Zhao, Y. Li, C. Chen, Biomaterials 2010, 31, 7606
29. R. C. Murdock, L. Braydich-Stolle, A. M. Schrand, J. J. Schlager, S. M. Hussain, Toxicol. Sci. 2008, 101, 239
30. L. F. Capitan-Vallvey, M. C. Moreno-Bondi, M. P. Marco, J. C. Sanchez-Lopez, I. S. Anderson, Crit. Rev. Solid State 2014, 39, 423.
31. Mislick K A, Baldeschwieler J D. Evidence for the role of proteoglycans in cation-mediated gene transfer. Proc Natl Acad Sci USA 1996; 93:12349e54.
32. Panyam J, Labhasetwar V. Biodegradable nanoparticles for drug and gene delivery to cells and tissue. Adv Drug Del Rev 2003; 55:329e47
33. Leroueil P R, Hong S, Mecke A, Baker J R, Orr B G, Holl M M B. Nanoparticle interaction with biological membranes: does nanotechnology present a Janus face? Acc Chem Res 2007; 40:335e42.
34. Hoekstra D, Rejman J, Wasungu L, Shi F, Zuhorn I. Gene delivery by cationiclipids: in and out of an endosome. Biochem Soc Trans 2007; 35:68e71.
35. M. N. Padamwar and V. B. Pokharkar, “Development of vitamin loaded topical liposomal formulation using factorial design approach: drug deposition and stability,” International Journal of Pharmaceutics, vol. 320, no. 1-2, pp. 37-44, 2006.
36. L. Mu and R. L. Sprando, “Application of nanotechnology in cosmetics,” Pharmaceutical Research, vol. 27, no. 8, pp. 1746-1749, 2010.
37. Wisuitiprot W, Somsiri A, Ingkaninan K, Waranuch N. In vitro human skin permeation and cutaneous metabolism of catechins from green tea extract and green tea extract-loaded
38. Pedro A S, Cabral-Albuquerque E, Ferreira D, Sarmento B. Chitosan: An option for development of essential oil delivery systems for oral cavity care. Carbobydr Polym, 2009; 76(4):501-8.
39. L'Oreal. 1998. Composition comprising an aqueous dispersion of lipid vesicles encapsulating a UV screening agent with acidic functionality and uses in topical application. U.S. Pat. No. 5,759,526 A.
40. Lacerda S P, Cerize N N P, Re M I. Preparation and characterization of carnauba wax nanostructured lipid carriers containing benzophenone-. Int J Cosmet Sci, 2011; 33:312-21.
41. Martins I, Barreiro M, Coelho M, Rodrigues A. Microencapsulation of essential oils with biodegradable polymeric carriers for cosmetic applications. Chem Eng J, 2014; 245:191-200
42. Mishra N, Goyal Khatri K. Biodegradable polymer based particulate carrier(s) for the delivery of proteins and peptides. Anti-Inflamm Anti-Allergy Agents Med Chem, 2008; 7:240-51.
43. Lee M H, Oh S G, Moon S K, Bae S Y. Preparation of silica particles encapsulating retinol using O/W/O multiple emulsions. J Colloid Interface Sci, 200; 240:83-89.

1. Anti-wrinkle and anti-aging nano-formulations for topical application to wrinkled skin tissue in the skin of a subject, wherein said nanoformulation is delivered using a nanosystem delivery that is penetrating the skin barrier using a specific mechanism, and wherein, the formulation releases the active substances in a controlled manner.

2. A method to prepare an anti-wrinkle and anti-aging nanoformulation, wherein, according to claim 1, wherein said the active ingredients are multi encapsulated such as: in a core silica biocompatible shell, followed by the co-encapsulation of other active ingredients using a polysaccharide, protein or a biodegradable polymer that has it's surface modified by cell penetrating peptides.

3. An anti-wrinkle and anti-aging nanoformulation composition according to claim 1, wherein said the active ingredients are a composition comprising essential oil and Argireline, aspartic acid, wherein the amount of active ingredients and oils in the final composition vary from about 0.1% (v/w) to about 30%(v/w) but most preferable from about 5% (v/w) to about 15% (v/w).

4. An anti-wrinkle and anti-aging nanoformulation composition according to claim 1, wherein the composition will additionally contain antimicrobial agents, wherein, the antimicrobial agents are green tea extracts, wherein they may be found in a concentration between 0.002% (v/w) to about 0.06% (v/w) but most preferable between 0.0125% (v/w) to about 0.0025% (v/w).

5. An anti-wrinkle and anti-aging nanoformulation composition according to claim 1, wherein the composition will additionally contain vitamins, wherein, the vitamins are selected from a group of: Vitamin C, Vitamin D, Vitamin K, Vitamin E, Vitamin, or/and a mixture of those, and where, the Vitamins concentration rage used is between 0.0005% (v/w) to about 0.01% (v/w) and most preferable from 0.001% (v/w) to about 0.005% (v/w).

6. An anti-wrinkle and anti-aging nanoformulation composition according to claim 1, wherein the composition will additionally contain minerals, wherein, the minerals (avoiding aluminum silicates) are selected as Mg Silicate, Zn, etc, where, they are used is a concentration rage between 0.0005% (v/w) to about 0.01% (v/w) but most preferable from about 0.001% (v/w) to about 0.005% (v/w).

7. An anti-wrinkle and anti-aging nanoformulation composition according to claim 1, wherein the composition will additionally contain moisturizer oils, wherein, the moisturizer oils are selected from a class of oils, as the case may be: pomegranate oil (Punica granatum), fennel oil (Foeniculum), citrus oil, rosemary oil (Rosmarinus officinalis), chamomile oil (Matricaria chamomilla), jojoba oil (Simmondsia chinensis), rosehip oil (Calendula officinalis), Geranium oil (Pelargonium graveolens) Lavender oil (Lavandula), tea-tree oil (Melaleuca alternifolia), Black cumin (Nigella saliva L.), coconut (Cocus nucifera), castor (Ricinus communis), safflower (Carthamus tinctorius), mustard (Brassica spp. and Sinapis alba), coriander, (Coriandrum sativum), squash (Cucurbita maxima), linseed/flax (Linum usitatissimum), Brazil nut (Bertholletia excelsa) jojoba and maize (Zea mays), and wherein, the amount of oils found in the final composition will vary between 1% (v/w) to about 60% (v/w), and most preferable from about 5% (v/w) to 40% (v/w).

8. An anti-wrinkle and anti-aging nanoformulation composition according to claim 1, wherein the composition comprises fragrances, typically from natural oils such as: coconut (Cocus nucifera), lavender (Lavandula), jasmine (Jasminum), floral, rose (Rosa), herbal, lily (Lilum), orange (Citrus X sinensis), etc, and wherein, the amount of fragrances vary between 0.005% (v/w) to about 10%(v/w), but most preferable between 0.5% (v/w) to about 5% (v/w).

9. An anti-wrinkle and anti-aging nanoformulation composition according to claim 1, wherein, the composition comprises anti-oxidants and other preservatives, and wherein, they may be Vitamin C, Optiphen Preservative, etc., in a concentration between 0.001% (v/w) to about 2% (v/w), but most preferable from 0.005% (v/w) to about 1% (WW).

10. An anti-wrinkle and anti-aging nanoformulation composition according to claim 1, wherein, the nanoformulation contain physically stabilizers used in a concentration of 0.01% (v/w) to about 2% (v/w), but most preferable between 0.005% to about 1% (v/w).

11. An anti-wrinkle and anti-aging nanoformulation composition according to claim 2, wherein, the nanocapsule contain multi layer shells comprise of: silica core shells, and polysaccharides (gums, starch, cellulose, cyclodextrines and chitosan), proteins (gelatin, casein and soy proteins), lipids (waxes, paraffin and oils).

12. An anti-wrinkle and anti-aging nanoformulation composition according to claim I, 2, wherein, the cell penetrating peptides are selected from a group of transactivation transcriptional activator protein (TAT) and polyarginine.

13. An anti-wrinkle and anti-aging nanoformulation composition according to claim 12 wherein, TAT is Gly-Tyr-Arg-Lys-Lys-Arg-Arg-Gln-Arg-Arg-Arg-His-His-His-His-His-His.

14. An anti-wrinkle and anti-aging nanoformulation composition according to claim 1-13, wherein said the active substances has anti-wrinkle and anti-aging properties and diminishes the wrinkle, wherein the nanoformulation of Example 1. diminishes 60% of the wrinkles in 30 days, when applied twice daily.
